(19) United States
(12) Patent Application Publication
(10) Pub. No.: US 2023/0355933 A1
(43) Pub. Date: Nov. 9, 2023

(54) Medical Device with Composite Core Wire

(71) Applicant: BOSTON SCIENTIFIC SCIMED, INC., Maple Grove, MN (US)

(72) Inventors: Aldo Mora Alvarado, Alajuela (CR); Brice Lee Shireman, Maple Grove, MN (US); Joel M. WasDyke, Independence, MN (US)

(73) Assignee: BOSTON SCIENTIFIC SCIMED, INC., Maple Grove, MN (US)

(21) Appl. No.: 18/143,853

(22) Filed: May 5, 2023

Related U.S. Application Data
(60) Provisional application No. 63/339,139, filed on May 6, 2022.

(51) Int. Cl. A61M 25/09; A61M 25/00

(52) U.S. Cl. CPC A61M 25/09; A61M 25/0012; A61M 2025/09166; A61M 2025/09083; A61M 2025/09108

(57) ABSTRACT

Medical devices as well as methods for making and using medical devices are disclosed. An example medical device may include a core wire having a proximal shaft portion and a distal shaft portion coupled to the proximal shaft portion. The proximal shaft portion may include a first material. The distal shaft portion may include an inner shaft formed from a second material and an outer shell disposed along an outer surface of the inner shaft. The outer shell may be formed of the first material.

### CROSS-REFERENCE TO RELATED APPLICATIONS

This application claims the benefit of priority under 35 U.S.C. § 119 of U.S. Provisional Application No. 63/339,139, filed May 6, 2022, the entire disclosure of which is hereby incorporated by reference.

### TECHNICAL FIELD

The present disclosure pertains to medical devices, and methods for manufacturing medical devices. More particularly, the present disclosure pertains to medical devices with a composite core wire.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A medical device is disclosed. The medical device comprises: a core wire having a proximal shaft portion and a distal shaft portion coupled to the proximal shaft portion; wherein the proximal shaft portion includes a first material; wherein the distal shaft portion includes an inner shaft formed from a second material and an outer shell disposed along an outer surface of the inner shaft, the outer shell being formed of the first material; and a tip member disposed over a distal region of the distal shaft portion.

Alternatively or additionally to any of the embodiments above, the first material includes stainless steel.

Alternatively or additionally to any of the embodiments above, the second material includes a nickel titanium alloy.

Alternatively or additionally to any of the embodiments above, the second material includes a super elastic nickel titanium alloy.

Alternatively or additionally to any of the embodiments above, the proximal shaft portion includes a taper.

Alternatively or additionally to any of the embodiments above, the distal shaft portion includes a taper.

Alternatively or additionally to any of the embodiments above, the outer shell includes a taper.

Alternatively or additionally to any of the embodiments above, the tip member includes a polymer tip.

Alternatively or additionally to any of the embodiments above, the tip member includes a coil.

Alternatively or additionally to any of the embodiments above, the inner shaft is tubular.

Alternatively or additionally to any of the embodiments above, further comprising a rod extending within the inner shaft.

Alternatively or additionally to any of the embodiments above, the rod includes a radiopaque material.

A method for manufacturing a core wire is disclosed. The method comprises: contacting a first end region of a first shaft with a second end region of a second shaft; wherein the first shaft includes a first material; wherein the second shaft includes an inner region and an outer shell disposed along the inner region, the inner region including a second material, the outer shell including the first material; thermally bonding the first shaft to the second shaft; and grinding a portion of the second shaft to remove at least a portion of the outer shell.

Alternatively or additionally to any of the embodiments above, the first material includes stainless steel.

Alternatively or additionally to any of the embodiments above, the second material includes a nickel titanium alloy.

Alternatively or additionally to any of the embodiments above, the second material includes a super elastic nickel titanium alloy.

Alternatively or additionally to any of the embodiments above, the second shaft has a third end region opposite the second end region and wherein grinding a portion of the second shaft to remove the outer shell from at least a portion of the second shaft includes grinding the second shaft at a location adjacent to the third end region.

A guidewire is disclosed. The guidewire comprises: a core wire having a proximal shaft and a distal shaft coupled to the proximal shaft; wherein the proximal shaft includes stainless steel; wherein the proximal shaft has a first region with a first outer diameter; wherein the proximal shaft has a second region disposed distally of the first region and having a second outer diameter that is less than the first outer diameter; wherein the distal shaft includes an inner nickel-titanium alloy member and an outer stainless steel cladding; wherein the distal shaft includes a third region that is attached to the second region of the proximal shaft; wherein the distal shaft includes a fourth region disposed distally of the third region, the fourth region being free from outer shell; and a tip member disposed over the fourth region of the distal shaft.

Alternatively or additionally to any of the embodiments above, the second region of the proximal shaft, the third region of the distal shaft, or both include a taper.

Alternatively or additionally to any of the embodiments above, the tip member includes a polymer guidewire tip or a spring tip.

A medical device is disclosed. The medical device comprises: a core wire having a proximal shaft portion and a distal shaft portion coupled to the proximal shaft portion; wherein the proximal shaft portion includes a first material; wherein the proximal shaft portion has a first region with a first outer diameter; wherein the proximal shaft portion has a second region disposed distally of the first region and having a second outer diameter that is less than the first outer diameter; wherein the distal shaft portion includes an inner shaft formed from a second material and an outer shell disposed along an outer surface of the inner shaft, the outer shell being formed of the first material; wherein the distal shaft portion includes a third region that is attached to the second region of the proximal shaft portion; wherein the distal shaft portion includes a fourth region disposed distally of the third region, the fourth region being free from the outer shell; and a tip member disposed over the fourth region of the distal shaft portion.

Alternatively or additionally to any of the embodiments above, the first material includes stainless steel.

Alternatively or additionally to any of the embodiments above, the second material includes a nickel titanium alloy.

Alternatively or additionally to any of the embodiments above, the second material includes a super elastic nickel titanium alloy.

Alternatively or additionally to any of the embodiments above, the second region of the proximal shaft portion includes a taper.

Alternatively or additionally to any of the embodiments above, the third region of the distal shaft portion includes a taper.

Alternatively or additionally to any of the embodiments above, the tip member includes a polymer tip.

Alternatively or additionally to any of the embodiments above, the tip member includes a coil.

Alternatively or additionally to any of the embodiments above, the inner shaft is tubular.

Alternatively or additionally to any of the embodiments above, further comprising a rod extending within the inner shaft.

Alternatively or additionally to any of the embodiments above, the rod includes a radiopaque material.

A method for manufacturing a core wire. The method comprises: contacting a first end region of a first shaft with a second end region of a second shaft; wherein the first shaft includes a first material; wherein the second shaft includes an inner region and an outer shell disposed along the inner region, the inner region including a second material, the outer shell including the first material; thermally bonding the first shaft to the second shaft; and grinding a portion of the second shaft to remove the outer shell from at least a portion of the second shaft.

Alternatively or additionally to any of the embodiments above, the first material includes stainless steel.

Alternatively or additionally to any of the embodiments above, the second material includes a nickel titanium alloy.

Alternatively or additionally to any of the embodiments above, the second material includes a super elastic nickel titanium alloy.

Alternatively or additionally to any of the embodiments above, the second shaft has a third end region opposite the second end region and wherein grinding a portion of the second shaft to remove the outer shell from at least a portion of the second shaft includes grinding the second shaft at a location adjacent to the third end region.

A guidewire is disclosed. The guidewire comprises: a core wire having a proximal shaft and a distal shaft coupled to the proximal shaft; wherein the proximal shaft includes stainless steel; wherein the proximal shaft has a first region with a first outer diameter; wherein the proximal shaft has a second region disposed distally of the first region and having a second outer diameter that is less than the first outer diameter; wherein the distal shaft includes an inner nickel-titanium alloy member and an outer stainless steel cladding; wherein the distal shaft includes a third region that is attached to the second region of the proximal shaft; wherein the distal shaft includes a fourth region disposed distally of the third region, the fourth region being free from outer shell; and a tip member disposed over the fourth region of the distal shaft.

Alternatively or additionally to any of the embodiments above, the second region of the proximal shaft includes a taper.

Alternatively or additionally to any of the embodiments above, the third region of the distal shaft includes a taper.

Alternatively or additionally to any of the embodiments above, the tip member includes a polymer guidewire tip or a spring tip.

A medical device is disclosed. The medical device comprises: a core wire having a proximal shaft portion and a distal shaft portion coupled to the proximal shaft portion; wherein the proximal shaft portion includes a first material; wherein the distal shaft portion includes an inner shaft formed from a second material and an outer shell disposed along an outer surface of the inner shaft, the outer shell being formed of a material that is compatible with the first material; and a tip member disposed over a distal region of the distal shaft portion.

A medical device is disclosed. The medical device comprises: a core wire having a proximal shaft portion and a distal shaft portion coupled to the proximal shaft portion; wherein the proximal shaft portion includes a first material; wherein the distal shaft portion includes an inner shaft formed from a second material and an outer shell disposed along an outer surface of the inner shaft, the outer shell being formed of a material that is weld-compatible with the first material; and a tip member disposed over a distal region of the distal shaft portion.

A medical device is disclosed. The medical device comprises: a core wire having a proximal shaft portion and a distal shaft portion coupled to the proximal shaft portion; wherein the proximal shaft portion includes a first material; wherein the distal shaft portion includes an inner shaft formed from a second material and an outer shell disposed along an outer surface of the inner shaft, the outer shell being formed of a material that is thermal bond-compatible with the first material; and a tip member disposed over a distal region of the distal shaft portion.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is partial cross-sectional side view of an example medical device.

FIG. 2 is partial cross-sectional side view of an example medical device.

FIGS. 3-5 illustrate an example core wire and/or an example process for manufacturing a core wire.

FIG. 6 is partial cross-sectional side view of an example medical device.

FIGS. 7-8 illustrate an example core wire and/or an example process for manufacturing a core wire.

FIG. 9 is partial cross-sectional side view of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

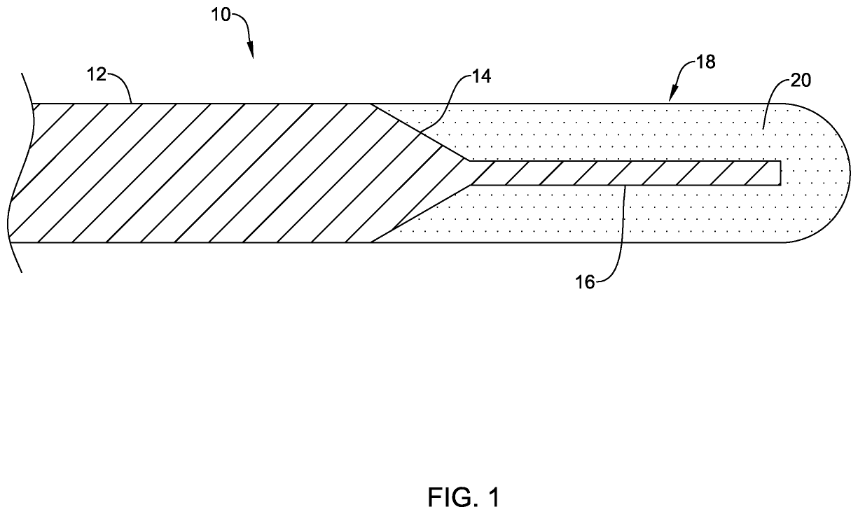

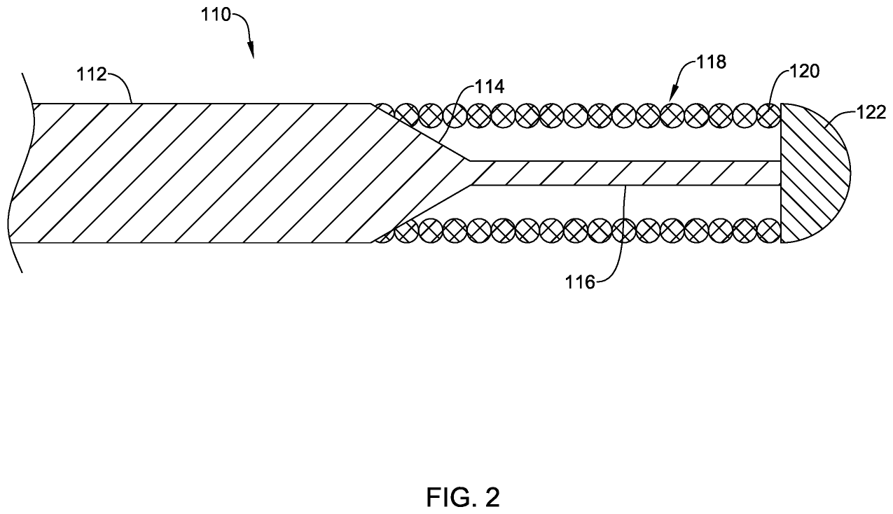

A wide variety of guidewires are known that can be used for a variety of interventions. For example, FIG. 1 schematically depicts an example guidewire 10. In this example, the guidewire 10 includes a core wire 12 with a tapered region 14 and a distal region 16. The guidewire 10 may include a distal tip 18. In this example, the distal tip 18 is a polymeric or “poly” tip that includes a polymeric tip member 20 disposed over the distal region 16. Similarly, FIG. 2 schematically depicts another example guidewire 110. In this example, the guidewire 110 includes a core wire 112 with a tapered region 114 and a distal region 116. The guidewire 110 may include a distal tip 118. In this example, the distal tip 118 is a “spring tip” includes a coil or spring 120 disposed over the distal region 116. A tip 122 (e.g., a solder ball tip) may be coupled to the coil 120.

In some instances, it may be desirable for the core wire 12, 112 to include structural features and/or materials that provide the guidewire 10, 110 with desirable characteristics. For example, it may be desirable for the more proximal portions of the guidewire 10, 110 to be stiffer and more distal portions of the guidewire 10, 110 to be more flexible. One way of accomplishing this general goal may include the use of tapers or reduced diameter regions of the core wire 12, 112, which may increase flexibility adjacent to the tapers/reduced diameter regions. In some of these and in other instances, it may be desirable to use different materials for different portions of the core wire 12, 112. Disclosed herein are core wires and/or guidewires using core wires (e.g., composite core wires) that include sections or portions formed from materials.

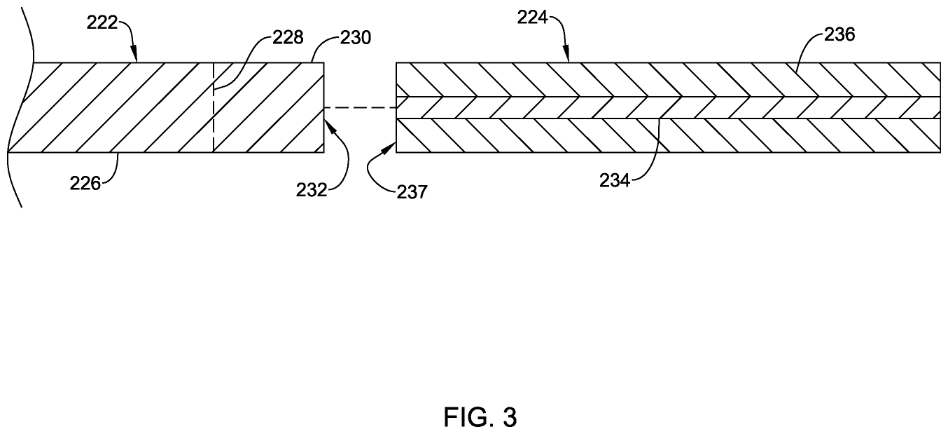

FIGS. 3-5 illustrate portions of a core wire, an example method for forming a core wire, and/or a core wire. In this example, the core wire is considered to be a composite core wire formed form multiple materials and/or sections that are joined together. For example, FIG. 3 illustrates a proximal shaft 222 and a distal shaft 224. The proximal shaft 222 may include a first region 226, a second region 230, and an end or end region 232. In FIG. 3, reference number 228 denotes an example position along the proximal shaft 222 where the first region 226 transitions to the second region 230. In some instances, reference number 228 may correspond to a location where the proximal shaft 222 will be processed or ground (e.g., as discussed herein). Also shown in FIG. 3 is a distal shaft 224. The distal shaft may include an inner shaft 234 and an outer shell or cladding 236. The distal shaft 224 may include an end or end region 237.

The proximal shaft 222 may be formed from or otherwise includes a first material. The first material may include one or more of the materials disclosed herein such as stainless steel. Stainless steel may be desirable for a number of reasons including, for example, providing a desirable level of stiffness and or pushability. The inner shaft 234 of the distal shaft 224 may include a second material. The second material may include one or more of the materials disclosed herein such as a nickel-titanium alloy. A nickel-titanium alloy may be desirable for a number of reasons including, for example, providing a desirable level of flexibility and/or elasticity. In some instances, the nickel-titanium alloy may be a shape memory and/or super elastic nickel titanium alloy. In other instances, the nickel-titanium alloy may be a linear elastic nickel titanium alloy. The outer shell 236 may include a material that is compatible with the first material, a material that is weld-compatible with the first material, a material that is thermal bond-compatible with the first material, the first material (e.g., stainless steel), and/or one or more of the materials disclosed herein.

The relative dimensions of the components of the distal shaft 224 may vary. It is noted that the dimensions are not necessarily shown to scale in the figures and should only be view as examples. For example, the inner shaft 234 may take up the majority of the volume of the distal shaft 224 whereas the outer shell 236 may contribute less to the volume of the distal shaft 224 than the inner shaft 234. In other instances, the outer shell 236 may take up the majority of the volume of the distal shaft 224. One way to quantify the relative contribution/dimensions would be to look at a cross-section of the distal shaft 224 and determine the relative area of the inner shaft 234 and/or the outer shell 236 relative to the total cross-sectional area of the distal shaft 224. In some instances, the distal shaft 224 may have a cross-sectional area and the inner shaft 234 may take up about 25% or more of the total cross-sectional area of the distal shaft 224, or about 40% or more of the total cross-sectional area of the distal shaft 224, or about 60% or more of the total cross-sectional area of the distal shaft 224, or about 75% or more of the total cross-sectional area of the distal shaft 224. These are just examples. Other dimensions are contemplated.

In examples where the proximal shaft 222 includes stainless steel and the distal shaft 224 (e.g., the inner shaft 234 of the distal shaft 224) includes a nickel titanium alloy, it may be challenging to thermally bond the proximal shaft 222 to the distal shaft 224. The inclusion of the outer shell 236 may help to improve the thermal bond compatibility between the proximal shaft 222 and the distal shaft 224. For example, when the outer shell 236 is formed from the same material as the proximal shaft 222 (or a material that has a favorable thermal bond compatibility with the material(s) used for the proximal shaft 222), it may be easier to form a resilient and reliable thermal bond between the proximal shaft 222 and the distal shaft 224. While not wishing to be bound by theory, it is believed that the thermal bond between the outer shell 236 and the proximal shaft 222 may help to enhance the bond between the proximal shaft 222 and the distal shaft 224, even if the inner shaft 234 has a lower thermal bond compatibility with the proximal shaft 222.

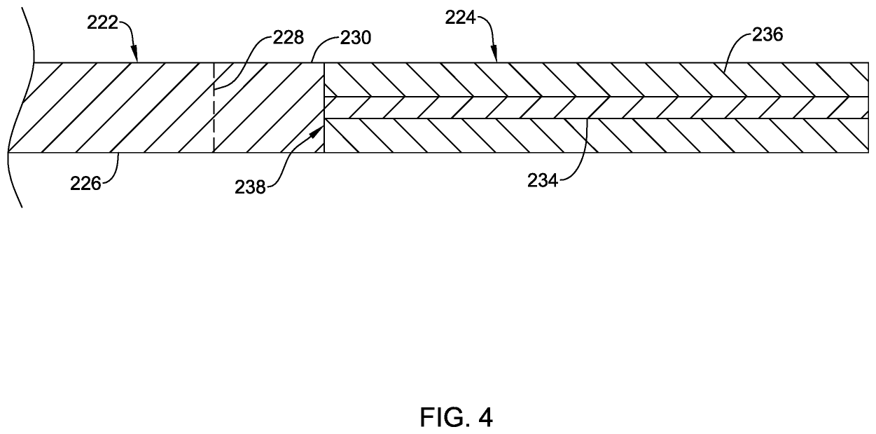

As shown in FIG. 4, the proximal shaft 222 may be brought into contact with the distal shaft 224. This may include forming a thermal bond (e.g., a weld) where the end 232 of the proximal shaft 222 is bonded to the end 237 of the distal shaft 224. In FIG. 4, the bonding/joining of the ends 232, 237 is marked by a joint 238.

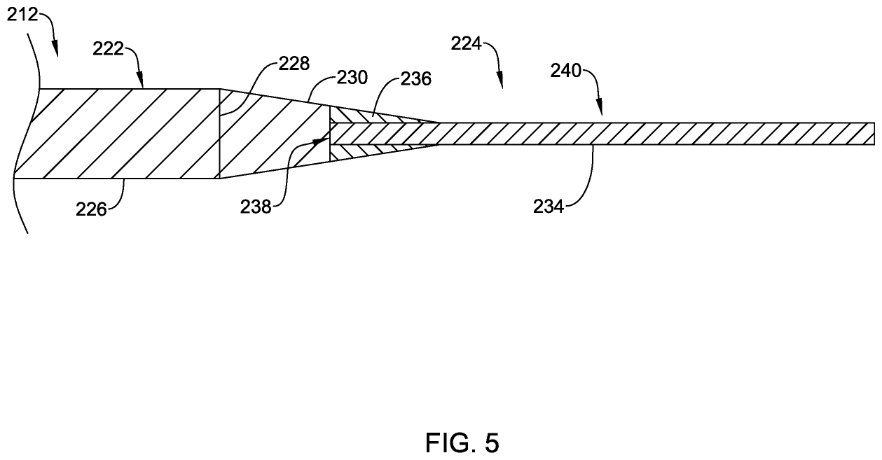

In some instances, proximal shaft 222 and/or the distal shaft 224 may be ground or otherwise processed to form a core wire 212 as shown in FIG. 5. In this example, the second region 230 of the proximal shaft 222 is ground to form/include one or more tapers and/or tapered regions. By doing so, the second region 230 (e.g., at least part of the second region 230) ends up having a reduced outer diameter relative to the first portion of the proximal shaft 222. The distal shaft 224 may also be ground. In this example, grinding may form a taper in the distal shaft 224. This may include removing a portion of the outer shell 236 and/or forming a taper in the outer shell 236. Grinding/tapering may continue until the outer shell 236 is tapered/removed to a point where the inner shaft 234 is exposed, thereby forming or defining a tip region 240 of the distal shaft 224. In some instances, the inner shaft 234 is also partially ground. In other instances, the inner shaft 234 remains unground. In this example, along the tip region 240 only the inner shaft 234 of the distal shaft 224 remains.

The dimensions of the core wire 212 after grinding may vary. For example, in some instances, the first region 226 may have a diameter of about 0.010-0.040 inches or less, 0.010-0.035 inches or less, or about 0.012-0.018 inches or less, or about 0.017 inches or less. The second region 230 may taper to an outer diameter of about 0.006-0.014 inches, or about 0.008-0.012 inches, or about 0.009-0.010 inches. The tip region 240 may have an outer diameter of about 0.004-0.010 inches, or about 0.005-0.007 inches, or about 0.006 inches. These are just examples.

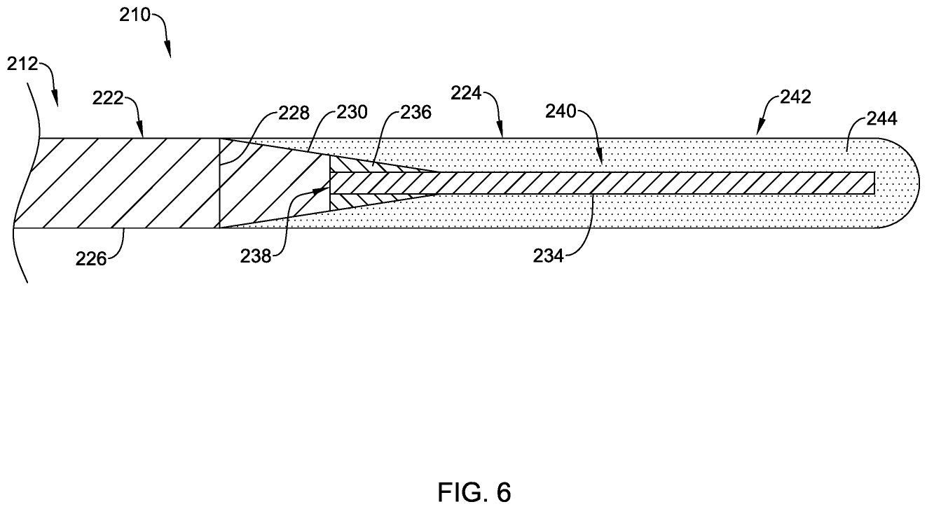

The core wire 212 may be used to form a guidewire 210 as shown in FIG. 6. The guidewire 210 may include a tip or tip member 242 is disposed along the tip region 240. In this example, the tip member 242 includes a polymer tip 244. Thus, the tip member 242 may be a polymer or “poly” tip similar to the schematic guidewire 10 shown in FIG. 1. In other examples, the tip member 242 may instead be a spring tip similar to that in the schematic guidewire 110 shown in FIG. 2. Other tips are contemplated including hybrid tips (e.g., that include both structures of polymer tips and structures of spring tips). In addition, other structures that may be used for guidewires may also be included in guidewire 210 such as shaping ribbons, marker bands, marker coils, and/or the like. Likewise, the core wire 212 may be used for a number of medical devices/structures including guidewires, catheters, leads, introducers, trocars, dilators, and/or the like. The guidewire 210 may be used for peripheral interventions, coronary interventions, delivering implants such as stents, prosthetic valves, etc.), and/or the like.

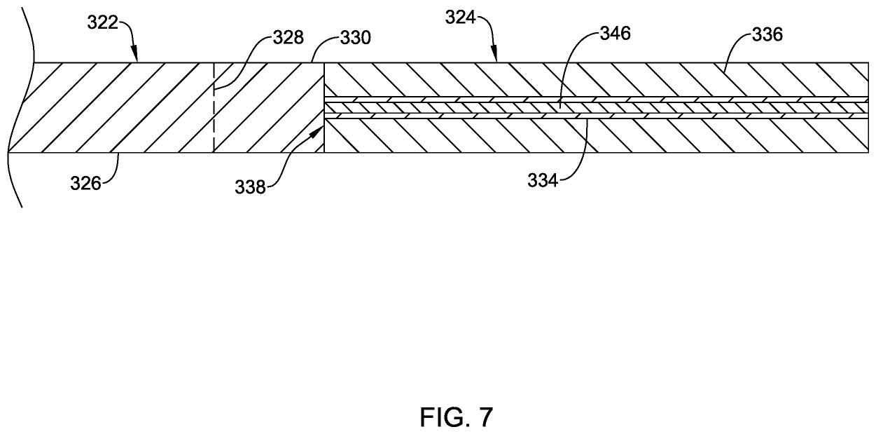

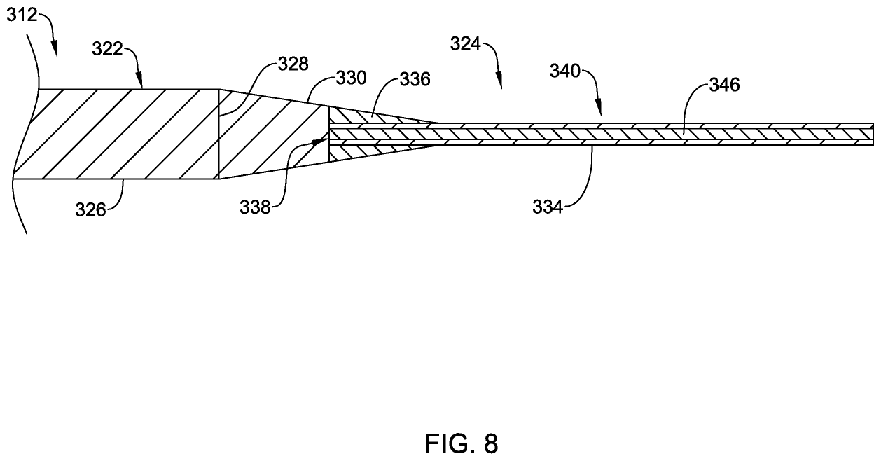

FIGS. 7-8 illustrate a portion of another core wire, an example method for forming another example core wire, and/or another example core wire. As shown in FIG. 7, the proximal shaft 322 may include a first region 326, a second region 330, and an end or end region. In FIG. 7, reference number 328 denotes an example position along the proximal shaft 322 where the first region 326 transitions to the second region 330. In some instances, reference number 328 may correspond to a location where the proximal shaft 322 will be processed or ground (e.g., as discussed herein). Also shown in FIG. 7 is a distal shaft 324. The distal shaft may include an inner shaft 334 and an outer shell or cladding 336. A rod 346 may extend through the inner shaft 334. The distal shaft 324 may include an end or end region. As shown in FIG. 7, a proximal shaft 222 may be brought into contact with a distal shaft 324. This may include attaching the end of the proximal shaft 322 to the end of the distal shaft 324. In FIG. 7, the joining of the ends is marked by a joint 338.

Just like the proximal shaft 222, the proximal shaft 322 may be formed from or otherwise includes a first material. The first material may include one or more of the materials disclosed herein such as stainless steel. Stainless steel may be desirable for a number of reasons including, for example, providing a desirable level of stiffness and or pushability. The inner shaft 334 of the distal shaft 324 may include a second material. The second material may include one or more of the materials disclosed herein such as a nickel-titanium alloy. A nickel-titanium alloy may be desirable for a number of reasons including, for example, providing a desirable level of flexibility and/or elasticity. In some instances, the nickel-titanium alloy may be a shape memory and/or super elastic nickel titanium alloy. In other instances, the nickel-titanium alloy may be a linear elastic nickel titanium alloy. The outer shell 336 may include compatible with the first material, a material that is weld-compatible with the first material, a material that is thermal bond-compatible with the first material, the first material (e.g., stainless steel), and/or one or more of the materials disclosed herein. In some instances, the rod 346 may include a radiopaque material (e.g., bismuth, tantalum, tungsten and/or tungsten alloys, gold, platinum, palladium, alloy iridium, combinations thereof, other materials such as those disclosed herein, and/or the like). In some of these and in other instances, the rod 346 may include stainless steel and/or a linear elastic nickel-titanium alloy. When the inner shaft 334 includes a super elastic and/or shape memory super elastic alloy, the use of a stainless steel and/or linear elastic nickel-titanium alloy rod 346 may help to provide some shapability to the guidewire 310 (e.g., shapability to the tip region 340).

In some instances, proximal shaft 322 and/or the distal shaft 324 may be ground or otherwise processed to form a core wire 312 as shown in FIG. 8. In this example, the second region 330 of the proximal shaft 322 is ground to form/include a taper. By doing so, the second region 330 ends up having a reduced outer diameter relative to the first portion of the proximal shaft 322. The distal shaft 324 may also be ground. Grinding/tapering may continue until the outer shell 336 is tapered/removed to a point where the inner shaft 334 is exposed, thereby forming or defining a tip region 340 of the distal shaft 324. Along the tip region 340, only the inner shaft 334 and the rod 346 remain. In some instances, the inner shaft 334 is also partially ground. In other instances, the inner shaft 334 remains unground.

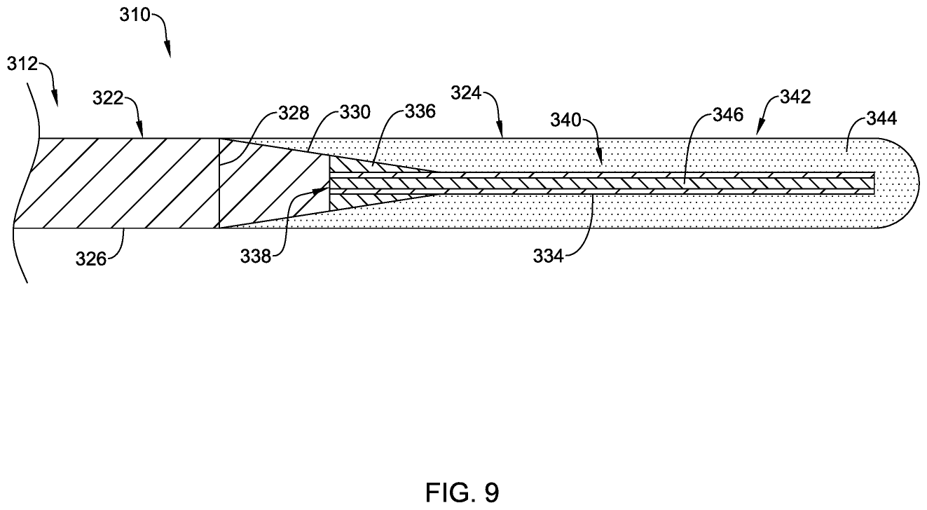

The core wire 312 may be used to form a guidewire 310 as shown in FIG. 9. The guidewire 310 may include a tip or tip member 342 is disposed along the tip region 340. In this example, the tip member 342 includes a polymer tip 344. Thus, the tip member 342 may be a polymer or “poly” tip similar to the schematic guidewire 10 shown in FIG. 1. In other examples, the tip member 342 may instead be a spring tip similar to that in the schematic guidewire 110 shown in FIG. 2. Other tips are contemplated including hybrid tips (e.g., that include both structures of polymer tips and structures of spring tips). In addition, other structures that may be used for guidewires may also be included in guidewire 310 such as shaping ribbons, marker bands, marker coils, and/or the like. Likewise, the core wire 312 may be used for a number of medical devices/structures including guidewires, catheters, leads, introducers, trocars, dilators, and/or the like. The guidewire 310 may be used for peripheral interventions, coronary interventions, delivering implants such as stents, prosthetic valves, etc.), and/or the like.

The materials that can be used for the various components of the guidewires 10, 110, 210, 310 may include those commonly associated with medical devices. For simplicity purposes, the following discussion makes reference to the guidewire 10 and other components of the guidewire 10. However, this is not intended to limit the devices and methods described herein, as the discussion may be applied to other similar tubular members and/or components of tubular members or devices disclosed herein.

The guidewire 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the guidewire 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the guidewire 10 to achieve the same result.

In some embodiments, a degree of Magnetic Resonance Imaging (Mill) compatibility is imparted into the guidewire 10. For example, the guidewire 10, or portions thereof, may be made of a material that does not substantially distort the image and create substantial artifacts (e.g., gaps in the image). Certain ferromagnetic materials, for example, may not be suitable because they may create artifacts in an MM image. The guidewire 10, or portions thereof, may also be made from a material that the Mill machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device, comprising:
a core wire having a proximal shaft portion and a distal shaft portion coupled to the proximal shaft portion;
wherein the proximal shaft portion includes a first material;
wherein the distal shaft portion includes an inner shaft formed from a second material and an outer shell disposed along an outer surface of the inner shaft, the outer shell being formed of the first material; and
a tip member disposed over a distal region of the distal shaft portion.

2. The medical device of claim 1, wherein the first material includes stainless steel.

3. The medical device of claim 1, wherein the second material includes a nickel titanium alloy.

4. The medical device of claim 1, wherein the second material includes a super elastic nickel titanium alloy.

5. The medical device of claim 1, wherein the proximal shaft portion includes a taper.

6. The medical device of claim 1, wherein the distal shaft portion includes a taper.

7. The medical device of claim 1, wherein the outer shell includes a taper.

8. The medical device of claim 1, wherein the tip member includes a polymer tip.

9. The medical device of claim 1, wherein the tip member includes a coil.

10. The medical device of claim 1, wherein the inner shaft is tubular.

11. The medical device of claim 10, further comprising a rod extending within the inner shaft.

12. The medical device of claim 11, wherein the rod includes a radiopaque material.

13. A method for manufacturing a core wire, the method comprising:
contacting a first end region of a first shaft with a second end region of a second shaft;
wherein the first shaft includes a first material;
wherein the second shaft includes an inner region and an outer shell disposed along the inner region, the inner region including a second material, the outer shell including the first material;
thermally bonding the first shaft to the second shaft; and
grinding a portion of the second shaft to remove at least a portion of the outer shell.

14. The method of claim 13, wherein the first material includes stainless steel.

15. The method of claim 13, wherein the second material includes a nickel titanium alloy.

16. The method of claim 13, wherein the second material includes a super elastic nickel titanium alloy.

17. The method of claim 13, wherein the second shaft has a third end region opposite the second end region and wherein grinding a portion of the second shaft to remove the outer shell from at least a portion of the second shaft includes grinding the second shaft at a location adjacent to the third end region.

18. A guidewire, comprising:
a core wire having a proximal shaft and a distal shaft coupled to the proximal shaft;
wherein the proximal shaft includes stainless steel;
wherein the proximal shaft has a first region with a first outer diameter;
wherein the proximal shaft has a second region disposed distally of the first region and having a second outer diameter that is less than the first outer diameter;
wherein the distal shaft includes an inner nickel-titanium alloy member and an outer stainless steel cladding;
wherein the distal shaft includes a third region that is attached to the second region of the proximal shaft;
wherein the distal shaft includes a fourth region disposed distally of the third region, the fourth region being free from outer shell; and
a tip member disposed over the fourth region of the distal shaft.

19. The guidewire of claim 18, wherein the second region of the proximal shaft, the third region of the distal shaft, or both include a taper.

20. The guidewire of claim 18, wherein the tip member includes a polymer guidewire tip or a spring tip.
